(19) United States
(12) Patent Application Publication
(10) Pub. No.: US 2024/0141108 A1
(43) Pub. Date: May 2, 2024

(54) POLYSUCCINIMIDE DERIVATIVE AND USE OF THE SAME FOR MAKING NANOMATERIAL

(71) Applicant: National Central University, Taoyuan City (TW)

(72) Inventor: Chun-Jen HUANG, Taoyuan City (TW)

(21) Appl. No.: 18/150,721

(22) Filed: Jan. 5, 2023

(30) Foreign Application Priority Data
Oct. 13, 2022 (TW) 111138795

(51) Int. Cl. C08G 73/10; A61K 9/51; C08G 73/14

(52) U.S. Cl. CPC C08G 73/1092; A61K 9/5146; C08G 73/1003; C08G 73/14

(57) ABSTRACT

Disclosed herein is a polysuccinimide derivative, which, under a pH of not greater than 6, includes a first repeating unit represented by formula (I), and a second repeating unit represented by formula (II),

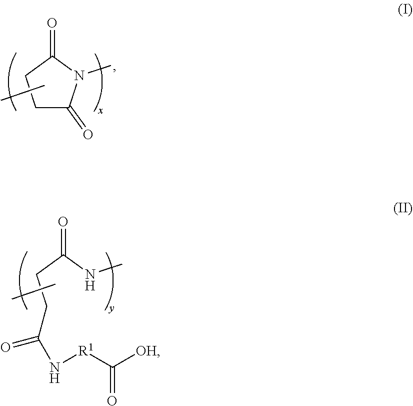

wherein each of the substituents is given the definition as set forth in the Specification and Claims. The second repeating unit is present in an amount ranging from 1 mol % to 90 mol % based on 100 mol % of the first repeating unit. Also disclosed herein is a nanomaterial including a plurality of nanoparticles, each of which, under a pH of not greater than 6, includes a hydrophobic substance and a carrier which is made from the polysuccinimide derivative and which encloses the hydrophobic substance.

### CROSS-REFERENCE TO RELATED APPLICATION

This application claims priority to Taiwanese Invention Patent Application No. 111138795, filed on Oct. 13, 2022.

### FIELD

The present disclosure relates to a polymer, and more particularly to a polysuccinimide derivative. The present disclosure also relates to use of the polysuccinimide derivative for making a nanomaterial.

### BACKGROUND

Carriers having pH-responsive property and with specific components, e.g., drugs or dyes, encapsulated therein, have been widely studied and utilized in various fields, e.g., disease treatment, antimicrobial dressings, indicators, etc., because such carriers are capable of controlling the release of the specific components through changes in pH of the environment.

Carrier having pH-responsive property are generally formed from amphiphilic polymer. For example, an article published in J. Control. Release, 2011, Vol. 152, p. 49-56, discloses pH-sensitive nanoparticles made from poly(ethylene gycol)-poly(L-histidine)-poly(L-lactide) (abbreviated as PEG-PH-PLLA) triblock copolymer. Each of the pH-sensitive nanoparticles includes an inner layer which is a hydrophobic PLLA segment, a middle layer which is a PH block structure having pH-responsive property, and an outer layer which is a hydrophilic PEG chain. The pH-sensitive nanoparticles with anti-cancer drugs encapsulated therein are used as carriers for anti-tumor drug delivery. The anti-cancer drugs would not be easily released from the pH-sensitive nanoparticles during transport of the pH-sensitive nanoparticles in the bloodstream that is weakly alkaline, e.g., having a pH value of 7.4, but would be released when the pH-sensitive nanoparticles reach the site of lesion that has a pH value lower than 7.0, e.g., pH value of 5.0. Since polysuccinimide has inherent biodegradable property, polysuccinimide has the potential to be developed as a pH-responsive carrier.

### SUMMARY

Therefore, in a first aspect, the present disclosure provides a polysuccinimide derivative which can alleviate at least one of the drawbacks of the prior art.

The polysuccinimide derivative, under a pH of not greater than 6, includes a first repeating unit represented by formula (I), and a second repeating unit represented by formula (II),

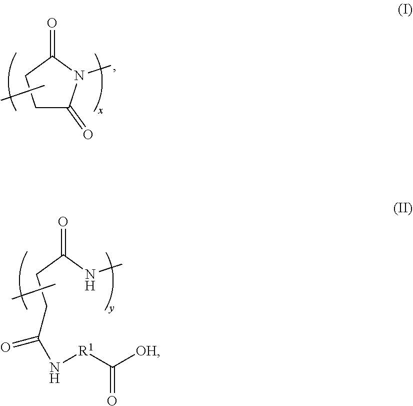

In formulas (I) and (II), x is an integer ranging from 5 to 1000, y is an integer ranging from 5 to 1000, and R¹ is selected from the group consisting of a C₁-C₂₀ straight chain alkyl group and a C₂-C₂₀ branched chain alkyl group. The second repeating unit is present in an amount ranging from 1 mol % to 90 mol % based on 100 mol % of the first repeating unit.

In a second aspect, the present disclosure provides a nanomaterial which can alleviate at least one of the drawbacks of the prior art.

The nanomaterial includes a plurality of nanoparticles. Under a pH of not greater than 6, each of the nanoparticles includes a hydrophobic substance and a carrier which is made from the aforesaid polysuccinimide derivative and which encloses the hydrophobic substance.

### BRIEF DESCRIPTION OF THE DRAWINGS

Other features and advantages of the present disclosure will become apparent in the following detailed description of the embodiment(s) with reference to the accompanying drawings. It is noted that various features may not be drawn to scale.

FIG. 1 shows nuclear magnetic resonance (NMR) spectra of molecular structures of polysuccinimides and polysuccinimide derivatives of Synthetic Examples 1 to 3.

FIG. 2 is a partially enlarged view of the NMR spectra shown in FIG. 1, illustrating chemical shift signals at 4.51 ppm for the polysuccinimides and the polysuccinimide derivatives of Synthetic Examples 1 to 3.

FIG. 3 shows attenuated total reflection-Fourier-transform infrared spectroscopy (ATR-FTIR) spectra of functional groups of the molecular structures of the polysuccinimides and the polysuccinimide derivatives of Synthetic Examples 1 to 3.

FIG. 4 is a graph showing turbidity of the suspension solution for preparing carriers of Preparative Examples 2, 5 and 8 at different pH values.

FIG. 5 is a scanning electron microscope (SEM) image illustrating a modified gauze bandage of Application Example 4.

FIG. 6 is a partially enlarged view of the SEM image shown in FIG. 5.

FIG. 7 is an SEM image illustrating a modified gauze bandage of Application Example 5.

FIG. 8 is a partially enlarged view of the SEM image shown in FIG. 7.

FIG. 9 is an SEM image illustrating a modified gauze bandage of Application Example 6.

FIG. 10 is a partially enlarged view of the SEM image shown in FIG. 9.

FIG. 11 is an SEM image illustrating a non-modified gauze bandage of Application Example 1 which serves as a control.

FIG. 12 is a graph showing cumulative amount of rifampicin released at different times for modified gauze bandages of Application Examples 1 to 3 under pH values of 5.0 and 7.5.

### DETAILED DESCRIPTION

Before the present disclosure is described in greater detail, it should be noted that if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a polysuccinimide derivative, under a pH of not greater than 6, includes a first repeating unit represented by formula (I), and a second repeating unit represented by formula (II),

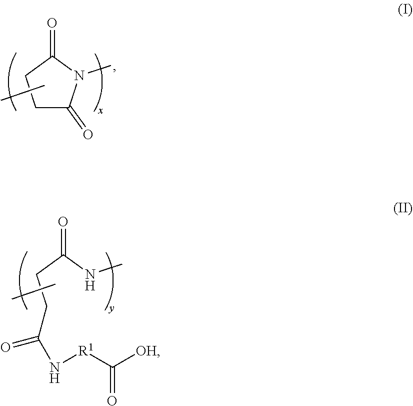

In formulas (I) and (II), x is an integer ranging from 5 to 1000, y is an integer ranging from 5 to 1000, and R¹ is selected from the group consisting of a C₁-C₂₀ straight chain alkyl group and a C₂-C₂₀ branched chain alkyl group. The second repeating unit is present in an amount ranging from 1 mol % to 90 mol % based on 100 mol % of the first repeating unit.

The polysuccinimide derivative may be used in subsequent applications, for example, to form nanoparticles by a precipitation process.

Therefore, the present disclosure also provides a nanomaterial which is a subsequent application of the polysuccinimide derivative. The nanomaterial includes a plurality of nanoparticles, and under a pH of not greater than 6, each of the nanoparticles includes a hydrophobic substance and a carrier which is made from the polysuccinimide derivative and which encloses the hydrophobic substance.

The polysuccinimide derivative, under a pH of not greater than 6, includes the first repeating unit that is hydrophobic and the second repeating unit that is both hydrophilic and hydrophobic, so the polysuccinimide derivative has both hydrophilic and hydrophobic properties, that is, the polysuccinimide derivative is an amphoteric polymer. By virtue of the polysuccinimide derivative being an amphoteric polymer, the carrier formed by self-assembly of the polysuccinimide derivative has a surface that is hydrophilic and an interior portion that is hydrophobic, thereby allowing the hydrophobic substance to be enclosed by the carrier.

The polysuccinimide derivative, under a pH of greater than 6, e.g., under a pH ranging from greater than 6 to not greater than 10, will be hydrolyzed so as to form a polyaspartic acid derivative including a third repeating unit represented by formula (III) and a fourth repeating unit represented by formula (IV),

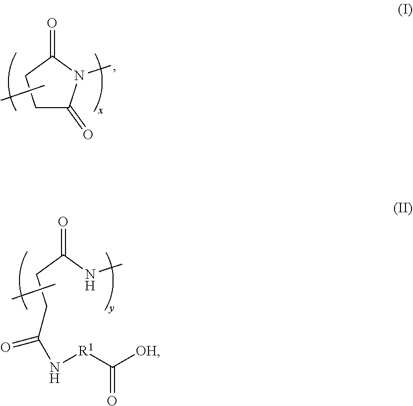

In formulas (III) and (IV), the definitions for x, y and R¹ are similar to those in formulas (I) and (II). To be specific, the more the number of carbon atom of R¹ in formula (II) is (i.e., nearing C₂₀), and/or the greater the amount of the second repeating unit is (i.e., nearing 90 mol %), the polysuccinimide derivative will be hydrolyzed in an environment having a pH that is near to 10, and the molecular structure thereof will change. Since the polyaspartic acid derivative is water-soluble and has biodegradable property, it is reasonable to infer that the polysuccinimide derivative is pH-responsive and has biodegradable property. In addition, the polysuccinimide derivative is hydrolyzed under a pH of greater than 6 so that molecular structure thereof is changed, i.e., when pH is increased from not greater to 6 to be greater than 6, the carrier made from the polysuccinimide derivative disintegrates in response to changes of the pH, thereby releasing the hydrophobic substance.

As used herein, the term “pH-responsive” can be used interchangeably with the term “pH-sensitive”, and is intended to mean that the molecular structure of a compound changes in response to changes in pH of a surrounding environment.

Since the polysuccinimide derivative is an amphoteric polymer, regardless of a hydrophilic and/or hydrophobic material (e.g., fiber in a fabric product), a physical force, e.g., van de Waals force, can be generated between the polysuccinimide derivative and the hydrophilic and/or hydrophobic material, and even between the hydrophilic and/or hydrophobic material and the nanomaterial which includes nanoparticles each including the carrier made from the polysuccinimide derivative, so that the polysuccinimide derivative and even the aforesaid nanomaterial will have good adhesion to the hydrophilic and/or hydrophobic material. Since the polysuccinimide derivative has good adhesion to the hydrophilic and/or hydrophobic material and since the polysuccinimide derivative is pH-responsive and has an inherent characteristic of biodegradable property, the polysuccinimide derivative and even the nanomaterial which includes nanoparticles each including the carrier made from the polysuccinimide derivatives are suitable for applications in various fields, such as biology and medicine, but are not limited thereto. For example, the polysuccinimide derivative and the nanomaterial may be attached to a material for medical use, e.g., gauze bandage, but is not limited thereto.

In certain embodiments, the polysuccinimide derivative has a number average molecular weight ranging from 20000 g/mol to 60000 g/mol.

In certain embodiments, R¹ is selected from the group consisting of a C₅-C₂₀ straight chain alkyl group and a C₅-C₂₀ branched chain alkyl group, such that the second repeating unit that is both hydrophilic and hydrophobic can be conferred with greater hydrophobicity, and thus allowing the polysuccinimide derivative to have greater hydrophobicity, so that during preparation of the nanomaterial, the carrier made from the polysuccinimide derivative may easily enclose the hydrophobic substance.

The second repeating unit is present in an amount of at least 1 mol % based on 100 mol % of the first repeating unit. In comparison to polysuccinimide, the polysuccinimide derivative is more sensitive to the changes in pH and responds faster, and the greater the content of the second repeating unit, the more the content of the hydrophobic substance enclosed by the carrier made from the polysuccinimide derivative is, and the faster the release of the hydrophobic substance under a pH of greater than 6 is. The content of the second repeating unit is not greater than 90 mol % based on 100 mol % of the first repeating unit, so that the carrier is prevented from disintegrating too quickly under a pH of greater than 6, thereby preventing the hydrophobic substance to be released too quickly. In certain embodiments, the second repeating unit is present in an amount ranging from 5 mol % to 25 mol % based on 100 mol % of the first repeating unit.

In certain embodiments, the hydrophobic substance is selected from the group consisting of a hydrophobic drug and a hydrophobic dye. In an exemplary embodiment, the hydrophobic drug is an antibiotic.

In certain embodiments, the carrier has an average particle size ranging from 20 nm to 1000 nm. In certain embodiments, the average particle size of the carrier ranges from 55 nm to 225 nm.

In certain embodiments, the nanoparticles each has an average particle size ranging from 20 nm to 1000 nm. It should be noted that, when the carrier encloses the hydrophobic substance to form each of the nanoparticles, certain forces occurring from, e.g., hydrophobic bonding, ionic bonding, hydrogen bonding, etc., will cause each of the nanoparticles to have a relatively small average particle size, and thus, it is possible and reasonable that the average particle size of the nanoparticles is smaller than the average particle size of the carrier itself (i.e., not enclosing the hydrophobic substance). In certain embodiments, the nanoparticles each has an average particle size ranging from 100 nm to 200 nm.

The present disclosure will be described by way of the following examples. However, it should be understood that the following examples are intended solely for the purpose of illustration and should not be construed as limiting the present disclosure in practice.

### EXAMPLES

### Preparation of Polysuccinimide Derivative

### Synthetic Example 1 (SE1)

The procedures for preparing the polysuccinimide derivative of SE1 includes the following steps A and B.

In step A, first, 12.5 mg of L-aspartic acid (purchased from Sigma-Aldrich), 1.25 mL of o-phosphoric acid, and 40 mL of a solvent formed by mixing 28 mL of 1,3,5-trimethylbenzene (i.e., mesitylene) and 12 mL of cyclobutane (i.e., sulfolane) were mixed to obtain a mixture. Next, using a Dean-Stark apparatus (i.e., a distilling trap), the mixture was subjected to a reaction by stirring under a nitrogen atmosphere at a temperature of 200° C. for 5 hours, so as to form a reactant. Afterwards, the reactant was cooled to a temperature of 25° C., and the solvent present in the reactant was removed via distillation under reduced pressure, so as to obtain a concentrated product. Then, the concentrated product, after being dissolved in 50 mL of dimethylformamide, was collectively added to 500 mL of methanol, so as to collect a resultant precipitate. In order to remove residual o-phosphoric acid in the precipitate, the precipitate was washed with distilled water until pH thereof was 6.0, thereby obtaining a crude product. Finally, the crude product was dried under vacuum to obtain polysuccinimide in powder form which has a total weight of 11.2 mg and a number average molecular weight of 38828 g/mol, with a yield of 89.6%.

In step B, 485.35 g (100 mol %) of the polysuccinimide obtained in step A, 10 mL of dimethylformamide, and 50.35 mg (5 mol %) of 11-aminoundecanoic acid (purchased from Sigma-Aldrich) were mixed to form a reactant, which were then subjected to stirring under a nitrogen atmosphere at a temperature of 85° C. for 48 hours, so as to form a polymeric component. Thereafter, the polymeric component was added to 100 mL of methanol, and the resultant polymer precipitate was washed several times with deionized water, followed by freeze-drying, so as to obtain a polysuccinimide derivative of SE1 in the form of powdered solids, which has a total weight of 408.35 mg and a number average molecular weight of 41848 g/mol, with a yield of 77.94%. The polysuccinimide derivative of SE1 includes a first repeating unit represented by formula (I) and a second repeating unit represented by formula (II),

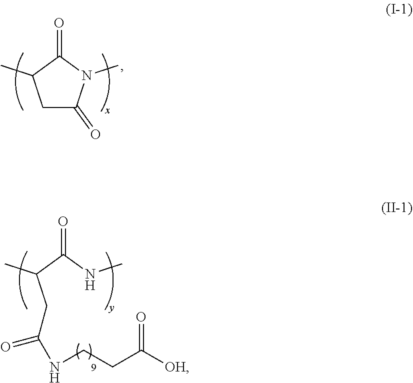

wherein in formulas (I) and (II), x is 385, and y is 15.

### Synthetic Examples 2 to 3 (SE2 to SE3)

The procedures and conditions for preparing the polysuccinimide derivatives of SE2 and SE3 were substantially similar those of SE1, except that in in step B, 11-aminoundecanoic acid is present in an amount of 10 mol % based on 100 mol % of the polysuccinimide in SE2, while 11-aminoundecanoic acid is present in an amount of 25 mol % based on 100 mol % of the polysuccinimide in SE3.

The thus obtained polysuccinimide derivative of SE2 has a total weight of 441.00 mg and a number average molecular weight of 44867 g/mol, with a yield of 78.56%. The polysuccinimide derivative of SE2 includes a first repeating unit represented by formula (I) and a second repeating unit represented by formula (II),

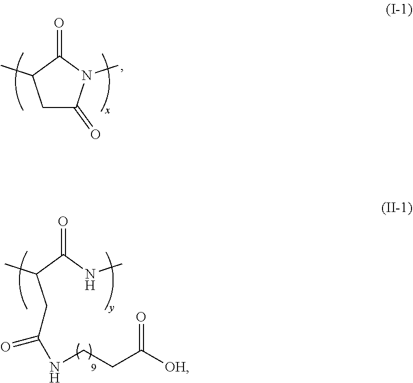

wherein in formulas (I) and (II), x is 370, and y is 30.

The thus obtained polysuccinimide derivative of SE3 has a total weight of 447.60 mg and a number average molecular weight of 53322 g/mol, with a yield of 66.95%. The polysuccinimide derivative of SE3 includes a first repeating unit represented by formula (I) and a second repeating unit represented by formula (II),

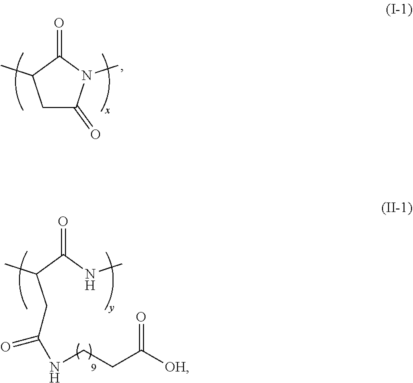

wherein in formulas (I) and (II), x is 328, and y is 72.

### Property Evaluation

### 1. Molecular Structure

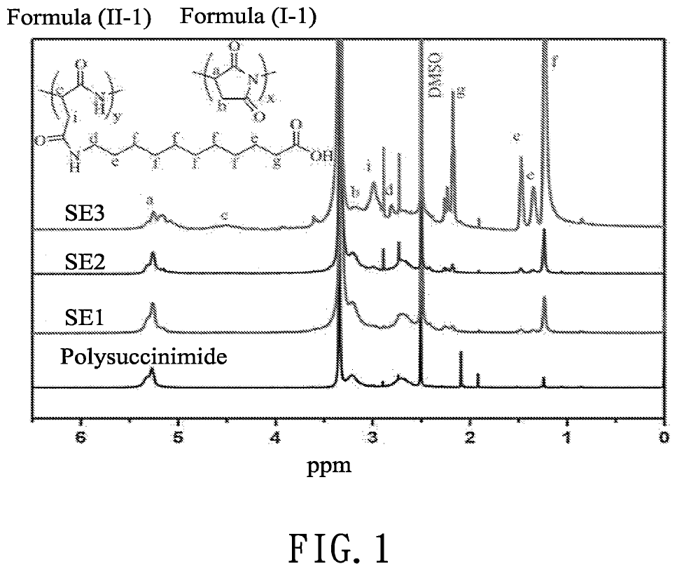

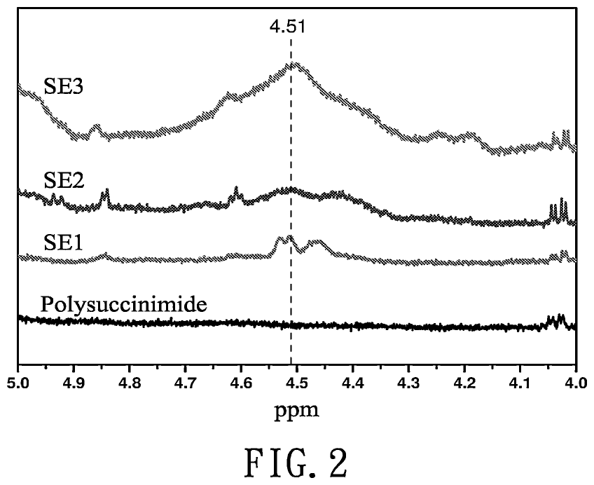

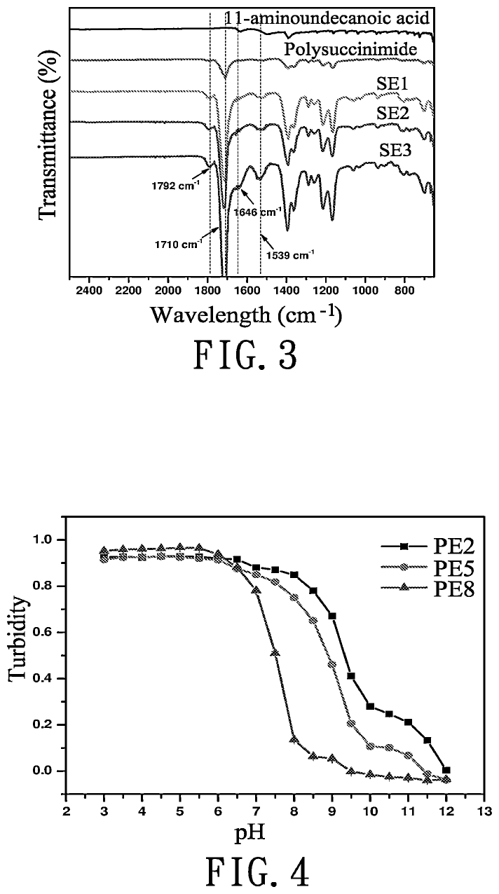

The polysuccinimides and polysuccinimide derivatives of SE1 to SE3 were analyzed to determine molecular structures thereof using a nuclear magnetic resonance (NMR) spectrometer (Manufacturer: Bruker Corporation; Model: Bruker Avance III HD 600 MHz). The results, presented as 1^{H}-NMR (DMSO-d₆) spectra, are shown in FIGS. 1 and 2. In addition, the polysuccinimide and the polysuccinimide derivatives of SE1 to SE3 were subjected to analysis of molecular structure of functional groups using attenuated total reflection-Fourier-transform infrared (ATR-FTIR) spectrometer (Manufacturer: Shimadzu Corporation; Model: IRSpirit) at a wavelength ranging from 4000 cm⁻¹ to 650 cm⁻¹. The results, presented as ATR-FTIR spectra, are shown in FIG. 3.

### 2. Number Average Molecular Weight of Polysuccinimide

First, 97.07 mg of each of the polysuccinimides of SE1 to SE3 was mixed with 10 mL of aqueous sodium hydroxide solution (0.1 M), such that the polysuccinimide was hydrolyzed to form sodium polyaspartate that is water soluble, followed by filtration using a filter having a pore size of 0.22 μm, so as to obtain a test sample. Next, the test samples was subjected gel permeation chromatography using GPC/SEC system including a A3000-single pore GPC/SEC column (300×8 mm) and a reflective index detector (Manufacturer: Malvern Panalytical Ltd.; Model: Viscotek VE3580), in which the test sample was analyzed at a temperature of 40° C. and a flow rate of 1 mL/min with water as the mobile phase, so as to obtain the number average molecular weight of sodium polyaspartate, which was then converted to obtain the number average molecular weight using the following Equation (I):

Number average molecular weight of polysuccinimide (g/mol)=Mnₛₐₘₚₗₑ−(Mnₛₐₘₚₗₑ÷Mn_{PAspNam})(Mnₐₘ) (I)

in which
Mnₛₐₘₚₗₑ=Number average molecular weight of sodium polyaspartate
Mn_{PAspNam}=Number average molecular weight of the monomer of sodium polyaspartate (i.e. 137.07 g/mol)
Mnₐₘ=Number average molecular weight of sodium hydroxide (i.e., 39.99 g/mol)

### 3. Yield of Polysuccinimide

The yield of polysuccinimide obtained in step A for preparing the polysuccinimide derivative of SE1 was calculated using the following Equation (II):

Yield of polysuccinimide (%)=W_{PSI}÷(n_{Asp}×Mn_{Asp})×100% (II)

in which
W_{PSI}=Weight of polysuccinimide (g)
n_{Asp}=Amount of L-aspartic acid in moles (mol)
Mn_{Asp}=Number average molecular weight of L-aspartic acid (i.e., 133.11 g/mol)

### 4. Content of Second Repeating Unit

Referring to FIGS. 1 and 2, the content of the first repeating unit was determined based on the integrated peak area of chemical shift signal at 5.27 ppm, while the content of the second repeating unit was determined based on the integrated peak area of chemical shift signal at 4.51 ppm. The content of the second repeating unit in each of the polysuccinimide derivatives of SE1 to SE3 was calculated based on the following Equation (III):

Content of the second repeating unit (mol %)=(Integrated peak area of chemical shift signal at 4.51 ppm)÷(Integrated peak area of chemical shift signal at 5.27 ppm)×100% (III)

The results are shown in Table 1.

### 5. Grafting Yield of 11-Aminoundecanoic Acid

The grafting yield of 11-aminoundecanoic acid in Step B for preparing the polysuccinimide derivatives of SE1 to SE3 was calculated using the following Equation (IV):

Grafting yield of 11-aminoundecanoic acid (%)=Amount of the second repeating unit (mol %)÷Total amount of the second repeating unit (i.e., 100 mol %)×100% (IV)

The results are shown in Table 1.

### 6. Number of x in Formula (I-1) and Number of y in Formula (II-1)

The number of x in formula (I-1) representing the first repeating unit and the number of yin formula (II-1) representing the second repeating unit of the polysuccinimide derivatives of SE1 to SE3 were calculated using the following Equations (Va) and (Vb):

Number of x in formula (I-1)=(1−R)×(Mn_{PSI}÷Mn_{PSIm}) (Va)

Number of y in formula (II-1)=R×(Mn_{PSI}÷Mn_{PSIm}) (Vb)

in which
R=Grafting yield of 11-aminoundecanoic acid in preparation of polysuccinimide derivative (%)
Mn_{PSI}=Number average molecular weight of polysuccinimide (i.e., 38828 g/mol)
Mn_{PSIm}=Number average molecular weight of the monomer of polysuccinimide (i.e., 97.07 g/mol)
   The results are shown in Table 1.

### 7. Number Average Molecular Weight (Mn) of Polysuccinimide Derivative

Number average molecular weight of each of the polysuccinimide derivatives of SE1 to SE3 was calculated using the following Equation (VI):

Number average molecular weight (Mn) of polysuccinimide derivative (g/mol)=(Number of x in formula (I-1)×Mn_{PSIm})+(Number of y in formula (II-1)×Mn_{PAm}) (VI)

in which
Mn_{PSIm}=Number average molecular weight of the monomer of polysuccinimide (i.e., 97.07 g/mol)
Mn_{PAm}=Number average molecular weight of the monomer of polysuccinimide derivatibe (i.e., 298.38 g/mol)
   The results are shown in Table 1.

### 8. Yield of Polysuccinimide Derivative

The yield of each of the polysuccinimide derivatives of SE1 to SE3 was calculated using the following Equation (VII):

Yield of polysuccinimide derivative (%)=W_{PA}÷{n_{PSI}×[R×Mn_{PAm}+(1−R)×Mn_{PSIm}]}×100% (VII)

in which
W_{PA}=Weight of polysuccinimide derivative (g)
n_{PSI}=Amount of polysuccinimide in moles (mol)
R=Grafting yield of 11-aminoundecanoic acid in preparation of polysuccinimide derivative (%)
Mn_{PAm}=Number average molecular weight of the monomer of polysuccinimide derivative (i.e., 298.38 g/mol)
Mn_{PSIm}=Number average molecular weight of the monomer of polysuccinimide (i.e., 97.07 g/mol)
   The results are shown in Table 1.

### 9. Biodegradability

InterLab Polyseed™ Microbial BOD capsules (Manufacturer: Cole-Parmer; Catalogue no.: EW-53200-33) were mixed with 500 mL of a diluent prepared according to the procedures set forth in OECD 301C Guideline for Testing of Chemicals 2005, followed by stirring for 1 hour under exposure to air, so as to obtain an inoculum. Meanwhile, 97.07 mg of the polysuccinimide derivative of SE1 was hydrolyzed with 20 mL of aqueous sodium hydroxide solution (0.05 N), so as to obtain a pretreatment solution. Next, 3.75 mL of the pretreatment solution, 90 mL of the inoculum, and 210 mL of the diluent were mixed to obtain a mixture, in which the concentration of the polysuccinimide derivative was 60 mg/L. Thereafter, 300 mL of the mixture was incubated in an incubator (Manufacturer: Firstek Scientific Co. Ltd., Model no.: BTH 80/−20) at a temperature of 25° C. for 28 days, so as to obtain an experimental group. In addition, a control group was prepared using the aforesaid procedures and conditions, except that the mixture only contained 90 mL of the inoculum and 210 mL of the diluent without the pretreatment solution. Afterwards, the chemical oxygen demand (COD) of the experimental and control groups were measured using Rocker's COD Test System including a CR 25 COD detector (Manufacturer: Rocker Scientific Co. Ltd.), and the biodegradability of the polysuccinimide derivative of SE1 was calculated using the following Equation (VIII):

Biodegradability (%)=1−[(C₂₈−C_{b28})÷(C₀−C_{b0})]×100% (VIII)

in which
C₂₈ represents the COD value of the experimental group on day 28
C_{b28} represents the COD value of the control group on day 28
C₀ represents the initial COD value of the experimental group (i.e., the COD value on day 0)
C_{b0} represents the initial COD value of the control group (i.e., the COD value on day 0)
   The results are shown in Table 1.

### Results and Discussions

TABLE 1

| | | | SE1 | SE2 | SE3 |
|---|---|---|---|---|---|
| Reactant | Polysuccinimide | Amount (mg) | 485.35 | 485.35 | 485.35 |
| | | Amount mol %) | 100 | 100 | 100 |
| | 11-aminoundecanoic | Amount (mg) | 50.33 | 100.66 | 251.65 |
| | acid | Amount (mol %) | 5 | 10 | 25 |

| | | | | |
|---|---|---|---|---|
| Polysuccinimide | Weight (mg) | 408.35 | 441.00 | 447.60 |
| derivative | Number average molecular weight | 41848 | 44867 | 53322 |
| | (g/mol) | | | |
| | Amount of first repeating unit (mol %) | 96.17 | 92.45 | 81.80 |
| | Amount of second repeating unit (mol %) | 3.83 | 7.55 | 18.20 |
| | Grafting rate of 11-aminoundecanoic | 3.83 | 7.55 | 18.20 |
| | acid (%) | | | |
| | Number of x in formula (I-1) | 385 | 370 | 328 |
| | Number of y in formula (II-1) | 15 | 30 | 72 |
| | Yield (%) | 77.94 | 78.56 | 66.95 |
| | Biodegradability (%) | 14.22 | 18.52 | 19.45 |

Referring to FIGS. 1 and 2, the three main peaks of the polysuccinimide were indicated by chemical shift signals at 5.27 ppm, 3.21 ppm and 2.70 ppm, respectively. As shown in the NMR spectra of each of the polysuccinimide derivatives of SE1 to SE3, a decrease in the peak of chemical shift signal at 5.27 ppm was accompanied with a corresponding increase in the peak of the chemical shift signal at 4.51 ppm, indicating that a greater amount of the monomers of the polysuccinimide reacted with the 11-aminoundecanoic acid to form the second repeating units, that is, the content of the second repeating unit increased from the polysuccinimide derivatives of SE1 to SE3 (see FIG. 1).

Referring to FIG. 3, each of the polysuccinimide derivatives of SE1 to SE2 had a peak at 1646 cm⁻¹ attributed to C═O stretching and a peak at 1539 cm⁻¹ attributed to C—N stretching and N—H bending, indicating that the monomers of the polysuccinimide indeed reacted with the 11-aminoundecanoic acid to form the second repeating units each having an amide group; whereas the other peaks at 1792 cm⁻¹ and 1710 cm⁻¹ were attributed to C═O stretching, indicating the presence of the first repeating units each having an imide group in each of the polysuccinimide derivatives of SE1 to SE3. In addition, the peak signals at 1646 cm⁻¹ and 1539 cm⁻¹ increased with an increased amount of the 11-aminoundecanoic acid.

As shown in Table 1, the biodegradability of the polysuccinimide derivatives of SE1 to SE3 ranges from 14% to 20%, and the higher the content of the second repeating unit, the better the biodegradability thereof is.

Preparation of Carrier Made from Polysuccinimide Derivative

### Preparative Example 1 (PE1)

First, 25 mg of polysuccinimide derivative of SE1 was dissolved in 2.5 mL of dimethyl sulfoxide (DMSO) to obtain a polymer solution, in which the concentration of polysuccinimide derivative is 10 mg/mL. Next, the polymer solution was added to 10 mL of deionized water having a pH of 5.0 (because carbon dioxide in the air dissolved into the deionized water to form carbonic acid), followed by stirring for 2 hours under a speed of 600 rpm, so as to obtain a nanoparticles-containing solution. Thereafter, the nanoparticles-containing solution was transferred into a dialysis bag having a molecular weight cut-off ranging from 6 kDa to 8 kDa, followed by placing the dialysis bag into the deionized water having a pH of 5.0 for dialysis treatment, such that the solution containing nanoparticles was concentrated (i.e., having reduced volume) to obtain a dialyzed product. Subsequently, the dialyzed product was subjected freeze-drying treatment to obtain a plurality of carriers of PE1 having a total weight of 22 mg and an average particle size of 62.29±0.44 nm.

### Preparative Examples 2 to 9 (PE2 to PE9)

The carriers of PE2 to PE9 were prepared using procedures and conditions similar to those of PE1, except for the source and amount of the polysuccinimide derivative, and the concentration of the polysuccinimide derivative in the polymer solution, as shown in Table 2 below.

### Property Evaluation

### 1. Average Particle Size

The carriers of each of PE1 to PE9 were mixed with deionized water to form a suspension solution, in which the concentration of the carriers was 100 μg/mL. Afterwards, the suspension solution was subjected to dynamic light scattering (DLS) measurement using DSL particle size analyzer (Manufacturer: Brookhaven Instruments Corporation; Model no.: NanoBrook Omni) so as to determine average particle size of the carriers. The results are shown in Table 2 below.

2. pH-Responsive Property

The carriers of PE2, PE5 and PE8 were mixed with deionized water to form a suspension solution, which had a volume of 5 mL and in which the concentration of the carriers was 2.5 mg/mL. Thereafter, pH of the suspension solution was gradually adjusted using aqueous hydrochloric acid solution (0.1 N) and aqueous sodium hydroxide solution (0.1 N), with each pH value difference being 0.5. After that, the percentage of transmittance (unit: %) of the suspension solution, under a pH ranging from 3.0 to 12.0, was measured using an Ultraviolet (UV)-Visible (Vis) spectrophotometer (Manufacturer: Shimadzu Corporation; Model no.: UV-1900) at a wavelength of 600 nm, followed by calculating the turbidity of the suspension solution based on the transmittance thereof using the following Equation (IX):

Turbidity=(100×Percentage of transmittance)÷100 (IX)

The results are shown in FIG. 4.

### Results and Discussions

TABLE 2

| | | PE1 | PE2 | PE3 | PE4 | PE5 | PE6 | PE7 | PE8 | PE9 |
|---|---|---|---|---|---|---|---|---|---|---|
| Polysuccinimide | Source | SE1 | | | SE2 | | | SE3 | | |
| | Amount (mg) | 25 | 62.5 | 125 | 25 | 62.5 | 125 | 25 | 62.5 | 125 |
| DMSO | Amount (mL) | 2.5 | 2.5 | 2.5 | 2.5 | 2.5 | 2.5 | 2.5 | 2.5 | 2.5 |
| Polymer | Concentration of | 10 | 25 | 50 | 10 | 25 | 50 | 10 | 25 | 50 |
| solution | polysuccinimide | | | | | | | | | |
| | derivative | | | | | | | | | |
| | (mg/mL) | | | | | | | | | |
| Carrier | Average | 62.29 ± | 105.40 ± | 221.13 ± | 57.34 ± | 99.83 ± | 187.66 ± | 59.78 ± | 83.28 ± | 122.90 [symbol] |
| | particle size | 0.44 | 0.47 | 1.17 | 0.12 | 0.36 | 1.89 | 0.63 | 2.15 | 2.59 |
| | (nm) | | | | | | | | | |

[symbol]

indicates data missing or illegible when filed

Referring to Table 2, in Preparative Examples 1 to 9, by adjusting the concentration of the polysuccinimide derivative dissolved to form the polymer solution, the average particle size of the carriers may be adjusted accordingly. In addition, when the concentration of the polysuccinimide derivative dissolved to form the polymer solution was greater than 25 mg/mL, as the content of the second repeating unit in the polysuccinimide derivative increased, the aggregation of the carriers made from the polysuccinimide derivative became less obvious, resulting in the average particle size of such carriers being smaller.

The higher the turbidity of the suspension solution, the lower the solubility of the carriers mixed with the deionized water to form the suspension solution is. As shown in FIG. 4, during the initial measurement of turbidity under a pH of 3, the turbidity of the suspension solutions of PE2, PE5 and PE8 were 0.925, 0.915 and 0.952, respectively, indicating that the carriers of PE2, PE5 and PE8 have a very poor solubility in water under a pH of 3. Under a pH of 7, the turbidity of the suspension solutions of PE2, PE5 and PE8 were 0.881, 0.850 and 0.781, respectively, indicating that the turbidity of these suspension solutions were slightly decreased compared with those under a pH of 3. Under a pH of 8.5, the turbidity of the suspension solutions of PE2, PE5 and PE8 were 0.780, 0.651 and 0.064, respectively, indicating that the turbidity of these suspension solutions were significantly decreased compared with those under pH of 3 and pH of 7, in particular, the suspension solution of PE8 showed the greatest decrease in turbidity thereof. These results demonstrated that the carriers of PE2, PE5 and PE8 have pH-responsive property, and the more the content of the second repeating unit in the polysuccinimide derivative, the faster the decrease in the turbidity of the suspension solution is, indicating the greater the significance of the pH-responsive property is.

Preparation of Nanomaterial Including Hydrophobic Substance and Carrier Made from Polysuccinimide Derivative

### Example 1 (EX1)

First, 25 mg of the polysuccinimide derivative of SE1 was dissolved in 1 mL of dimethyl sulfide, so as to obtain a polymer solution, in which the concentration of the polysuccinimide derivative was 25 mg/mL. Next, 1.25 mg of rifampicin (i.e., an antibiotic that is hydrophobic and purchased from BioVision, Inc.) was mixed with the polymer solution to obtain a mixture, in which the weight ratio of rifampicin to the polysuccinimide derivative of SE1 was 0.05:1. Thereafter, the mixture was added to 10 mL of deionized water having a pH of 5.0, followed by stirring at a speed of 600 rpm for 2 hours, so as to obtain a nanoparticles-containing solution. Afterwards, the nanoparticles-containing solution was transferred into a dialysis bag having a molecular weight cut-off ranging from 6 kDa to 8 kDa, followed by placing the dialysis bag into the deionized water having a pH of 5.0 for dialysis treatment, followed by a freeze-drying process, so as to obtain a nanomaterial of EX1 which has a total weight of 18.41 mg and which includes a plurality of nanoparticles. Each of the nanoparticles includes the rifampicin and a carrier that is made from the polysuccinimide derivative of SE1 and that encloses the rifampicin.

### Examples 2 to 12 (EX2 to EX12)

The procedures and conditions for preparing the nanomaterials of EX2 to EX12 were substantially the same as those of EX1, except for the differences in the source of the polysuccinimide derivative and the amount of rifampicin, as shown in Table 3 below.

TABLE 3

| | | 1 | 2 | 3 | 4 | 5 | 6 | 7 | 8 | 9 | 10 | 11 | 12 |
|---|---|---|---|---|---|---|---|---|---|---|---|---|---|
| Polysuccinimide | Source | SE1 | | | | SE2 | | | | SE3 | | | |
| derivative | Amount | 25 | 25 | 25 | 25 | 25 | 25 | 25 | 25 | 25 | 25 | 25 | 25 |
| | (mg) | | | | | | | | | | | | |
| Rifampicin | Amount | 1.25 | 2.50 | 3.75 | 5.00 | 1.25 | 2.50 | 3.75 | 5.00 | 1.25 | 2.50 | 3.75 | 5.00 |
| | (mg) | | | | | | | | | | | | |
| Weight ratio of rifampicin to | | 0.05 | 0.1 | 0.15 | 0.2 | 0.05 | 0.1 | 0.15 | 0.2 | 0.05 | 0.1 | 0.15 | 0.2 |
| polysuccinamide derivative | | | | | | | | | | | | | |
| Nanomaterial | Amount | 18.41 | 19.25 | 20.13 | 21.00 | 13.63 | 14.27 | 14.92 | 15.57 | 19.68 | 20.63 | 21.56 | 22.50 |
| | (mg) | | | | | | | | | | | | |

### Application of Nanomaterial

### Application Example 1 (AE1)

First, 25 mg of the nanomaterial of EX3 was mixed with 1 mL of dimethyl sulfoxide (DMSO) to obtain a treatment solution. Next, a non-modified gauze bandage (Manufacturer: 3M Company) made from cotton and having a dimension of 3.5 cm×3.5 cm and white-colored was soaked in the treatment solution, and then placed in a refrigerator at 4° C. away from light for 1 day, so as to obtain a treatment solution-soaked gauze bandage. Thereafter, the treatment solution-soaked gauze bandage was subjected to a vacuum-drying process, followed by a washing process conducted several times with an aqueous acidic solution having a pH of 4.5 (prepared by mixing aqueous hydrochloric acid solution (0.01 N) and deionized water having a pH of 5.0) until wastewater from the washing process was free from the nanomaterial of EX3 including the rifampicin and the polysuccinimide derivative, as detected by UV-Vis spectrophotometry, so as to obtain a washed gauze bandage. Then, the washed gauze bandage was allowed to dry away from light, thereby obtaining a modified gauze bandage of AE1 which had an orange color.

Afterwards, the modified gauze bandage of AE1 was cut into two pieces of modified gauze bandages each having a dimension of 1.2 cm×1.2 cm. Then, one of the modified gauze bandages was placed into a first dialysis bag having a molecular weight cut-off ranging from 6 kDa to 8 kDa, followed by adding into the first dialysis bag, 15 mL of an ascorbic acid solution which was colorless and having a pH of 5.0, which was prepared by dissolving ascorbic acid (i.e., vitamin C) in a citric acid buffer solution having a pH of 5.0, in which the concentration of ascorbic acid was 200 μg/mL, so that the modified gauze bandage was soaked in the ascorbic acid solution having a pH of 5.0. Subsequently, the first dialysis bag was placed into another ascorbic acid solution having a pH of 5.0 for dialysis treatment, which was conducted under stirring at a temperature of 37° C. and a speed of 100 rpm for 48 hours, thereby obtaining, in the first dialysis bag, a first test sample of AE1 (i.e., the modified gauze bandage after the dialysis treatment) having an orange color and a first test liquid of AE1 (i.e., the ascorbic acid solution after the dialysis treatment) that was pale yellow to nearly colorless.

Meanwhile, the other one of the modified gauze bandages was placed into a second dialysis bag having a molecular weight cut-off ranging from 6 kDa to 8 kDa, followed by adding into the second dialysis bag, 15 mL of an ascorbic acid solution which was colorless and having a pH of 7.5, which was prepared by dissolving ascorbic acid in a phosphate buffer solution having a pH of 7.5, in which the concentration of ascorbic acid was 200 μg/mL, so that the modified gauze bandage was soaked in the ascorbic acid solution having a pH of 7.5. Subsequently, the second dialysis bag was placed into another ascorbic acid solution having a pH of 5.0 for dialysis treatment, which was conducted under stirring at a temperature of 37° C. and a speed of 100 rpm for 48 hours, thereby obtaining, in the second dialysis bag, a second test sample of AE1 (i.e., the modified gauze bandage after the dialysis treatment) having a white color and a second test liquid of AE1 (i.e., the ascorbic acid solution after the dialysis treatment) having a yellow color.

### Application Examples 2 and 3 (AE2 and AE3)

The procedures and conditions for preparing the first and second test samples and the first and second test liquids of AE2 and AE3 were substantially the same as those of AE1, except for the differences in the source of nanomaterial, as shown in Table 4 below.

### Application Examples 4 to 6 (AE4 to AE6)

The procedures and conditions for preparing the first and second test samples and the first and second test liquids of AE4 to AE6 were substantially the same as those of AE1, except for the differences in the source of nanomaterial. To be specific, the modified gauze bandage of AE4 was obtained by soaking a non-modified gauze bandage in a treatment solution prepared by mixing the nanomaterial of EX4 with DMSO; the modified gauze bandage of AE5 was obtained by soaking a non-modified gauze bandage in a treatment solution prepared by mixing the nanomaterial of EX8 with DMSO; and the modified gauze bandage of AE6 was obtained by soaking a non-modified gauze bandage in a treatment solution prepared by mixing the nanomaterial of EX12 with DMSO.

### Property Evaluation

### 1. Scanning Electron Microscopy

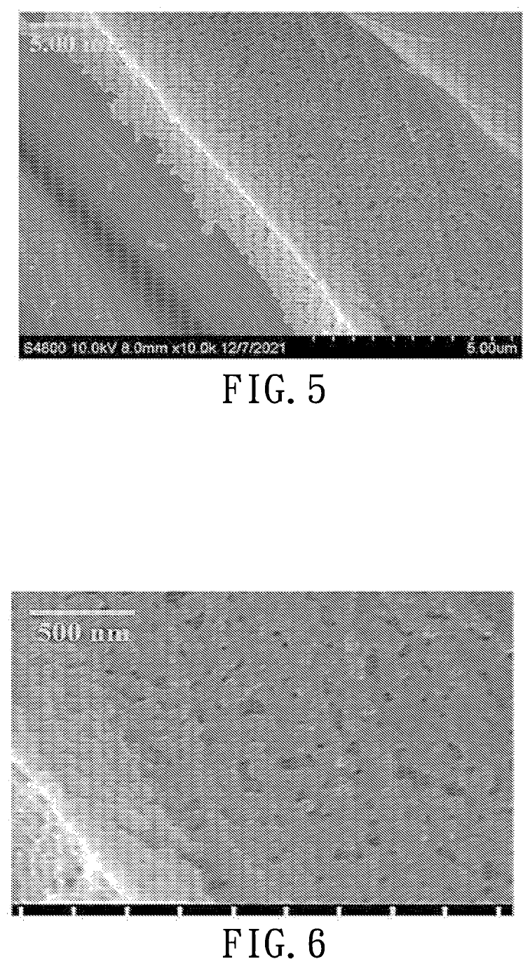

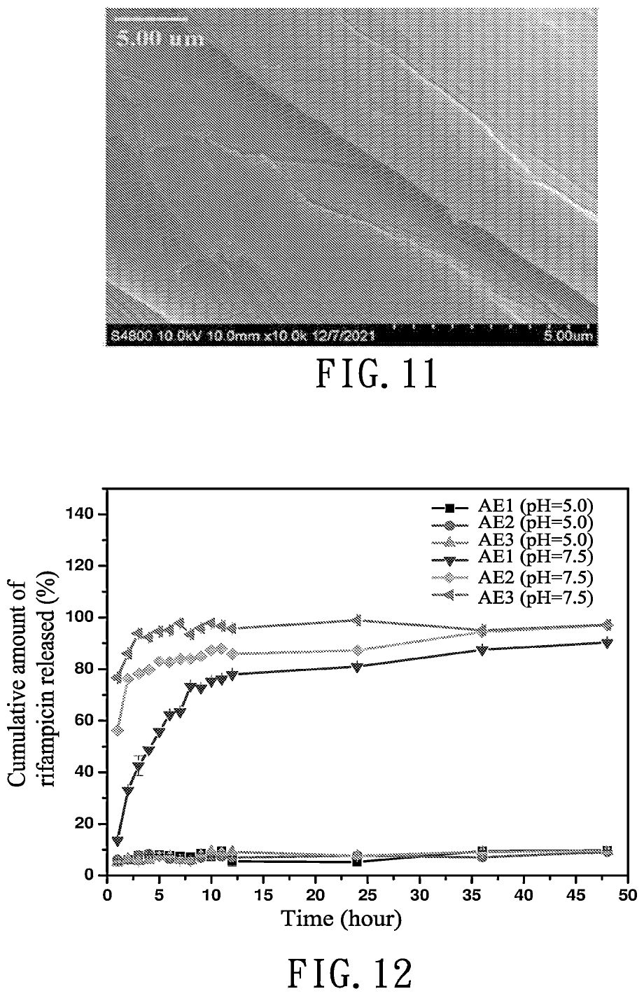

The modified gauze bandages of AE4 to AE6 were subjected to imaging using a scanning electron microscope (Manufacturer: Hitachi High-Tech Corporation; Model no.: SU8200) so as to observe external appearance thereof and to measure the average particle size of the nanomaterial in each of the modified gauze bandages of AE4 to AE6. The results are shown in FIGS. 5 to 10. In addition, the non-modified gauze bandage of AE1 was also subjected to aforesaid experimental procedures, and the results are shown in FIG. 11.

### 2. Cumulative Amount of Rifampicin Released

In each of AE1 to AE3, during the aforesaid dialysis treatment which was conducted for 48 hours, 2 mL of the first test liquid in the first dialysis bag and 2 mL of the second test liquid in the second dialysis bag were taken out at regular time intervals to be analyzed using an UV-Vis spectrophotometer (Manufacturer: Shimadzu Corporation; Model no.: UV-1900) at a wavelength of 475 nm, so as to measure the percentage of transmittance (unit: %) of each of the first and second test liquids, followed by calculating the cumulative amount of rifampicin released from the first and second test samples based on the transmittances thereof using the following Equations (Xa) and (Xb):

y₁=0.0181x₁−0.00269 (Xa)

y₂=0.0186x₂−0.00415 (Xb)

in which
y₁=transmittance of the first test liquid in the first dialysis bag (%)
x₁=cumulative amount of rifampicin released from the first test sample (%)
y₂=transmittance of the second test liquid in the second dialysis bag (%)
x₂=cumulative amount of rifampicin released from the second test sample (%)

The cumulative amounts of rifampicin released from each of the first test sample under pH 5.0 and the second test sample under pH 7.5 of AE1 to AE3 at different times during the dialysis treatment are shown as a graph in FIG. 12.

TABLE 4

| | AE1 | | AE2 | | AE3 | |
|---|---|---|---|---|---|---|
| Color of non-modified gauze | White | | White | | White | |
| bandage | | | | | | |
| Source of nanomaterial | EX3 | | EX7 | | EX11 | |
| Color of modified gauze | Orange | | Orange | | Orange | |
| bandage | | | | | | |
| Color of ascorbic acid | Colorless | | Colorless | | Colorless | |
| solution | | | | | | |
| pH of ascorbic acid solution | 5.0 | 7.5 | 5.0 | 7.5 | 5.0 | 7.5 |
| Color of first test sample | Orange | White | Orange | White | Orange | White |
| under pH 5.0 and second | | | | | | |
| test sample under pH 7.5 | | | | | | |
| Color of first test liquid under | Pale | Yellow | Pale | Yellow | Pale | Yellow |
| pH 5.0 and second test liquid | yellow to | | yellow to | | yellow to | |
| under pH 7.5 | nearly | | nearly | | nearly | |
| | colorless | | colorless | | colorless | |
| Cumulative amount of | 9.73 | 90.31 | 9.10 | 96.95 | 9.50 | 97.15 |
| rifampicin released after 48 | | | | | | |
| hours | | | | | | |

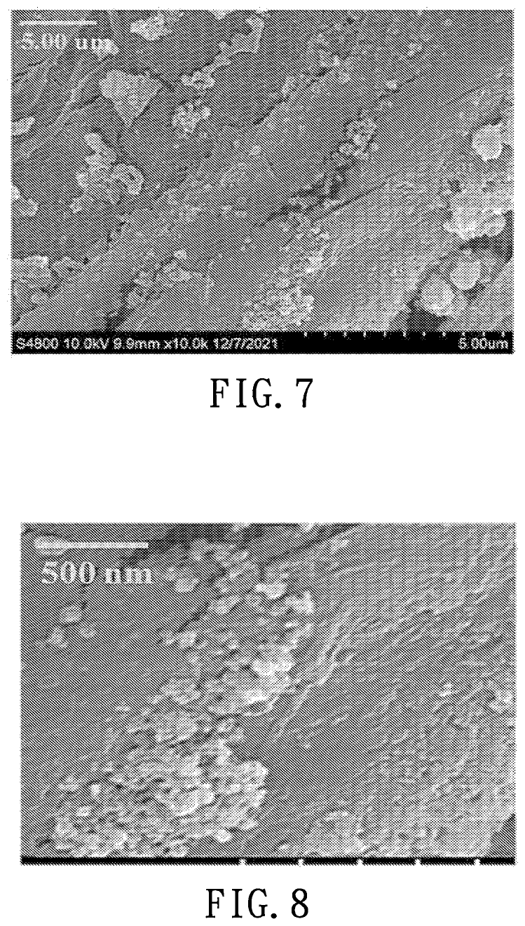

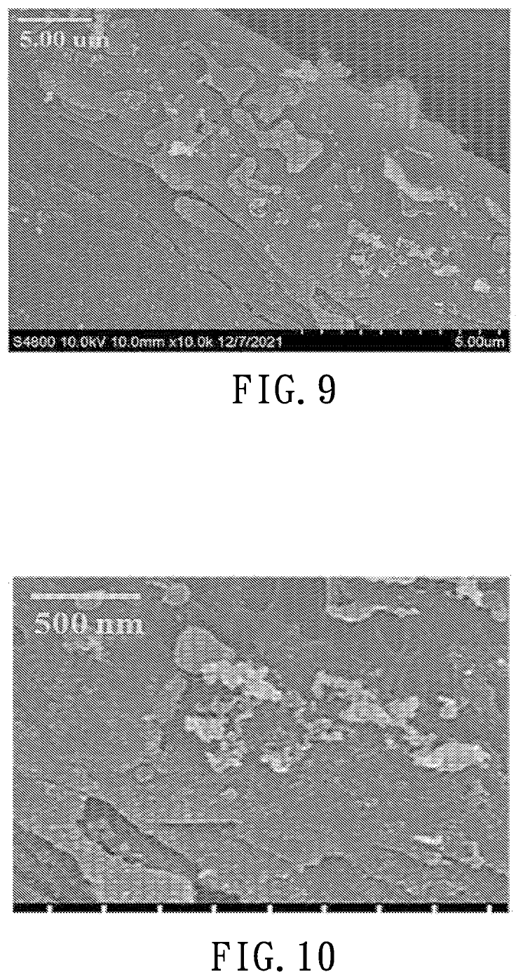

The non-modified gauze bandage of AE1 (see FIG. 11), which was not soaked in the treatment solution containing the nanomaterial, has a smooth surface, whereas the modified gauze bandage of AE4 (see FIGS. 5 and 6), the modified gauze bandage of AE5 (see FIGS. 7 and 8), and the modified gauze bandage of AE6 (see FIGS. 9 and 10), which were soaked in the treatment solutions containing nanomaterials of EX4, EX8 and EX12, respectively, have rough surfaces due to the presence of nanoparticles in the nanomaterials. In addition, the nanoparticles in the modified gauze bandages of AE4 to AE5 have an average particle size ranging from 100 nm to 200 nm.

Referring to FIG. 12 and Table 5, since only a slight amount of the nanomaterial in the modified gauze bandage of AE1 was dissolved in the ascorbic acid solution having a pH of 5.0, the first test sample of AE1 obtained after the dialysis treatment still had the same color as that of the modified gauze bandage (i.e., orange color), the first test liquid had a color (i.e., pale yellow to nearly colorless) substantially similar to that of the ascorbic acid solution (i.e., colorless), and the cumulative amount of rifampicin released from the modified gauze bandage into the ascorbic acid solution, as determined from the first test solution, was only 9.73%. In contrast, since a large amount of the nanomaterial in the modified gauze bandage of AE1 was dissolved in the ascorbic acid solution having a pH of 7.5, the second test sample of AE1 obtained after the dialysis treatment had the same color as that of the non-modified gauze bandage (i.e., white color), the second test liquid had a yellow color due to the release of rifampicin from the modified gauze bandage, and the cumulative amount of rifampicin released from the modified gauze bandage into the ascorbic acid solution, as determined from the second test solution, was as high as 90.31%. Similar results are obtained when the modified gauze bandages of AE2 and AE3 were subjected to the dialysis treatment after being soaked in the ascorbic acid solution having a pH of 5.0 and the ascorbic acid solution having a pH of 7.5. These results demonstrated that, each of the modified gauze bandages of AE1 to AE3 including the nanomaterial of the present disclosure has pH-responsive property (i.e., sensitive to change in pH), and rifampicin is easily released from each of the modified gauze bandages under a pH of 7.5, but not under a pH of 5.0.

It is known that healthy human skin has a pH ranging from 4 to 6, while bacteria-infected skin has a pH of greater than 7. The abovementioned results confirmed that, the modified gauze bandages of AE1 to AE3, due to the presence of nanomaterials therein, are capable of controlling the release of rifampicin therefrom under a pH of greater than 6, and thus, are suitable for treating bacteria-infected skin.

In summary, the polysuccinimide derivative of the present disclosure that includes the first repeating unit and the second repeating unit, allows the nanomaterial of the present disclosure, which includes the nanoparticles each including the hydrophobic substance and the carrier made from the polysuccinimide derivative, to release the hydrophobic substance therefrom when the pH changes from not greater than 6 to greater than 6.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A polysuccinimide derivative, under a pH of not greater than 6, comprising:
a first repeating unit represented by formula (I); and
a second repeating unit represented by formula (II), 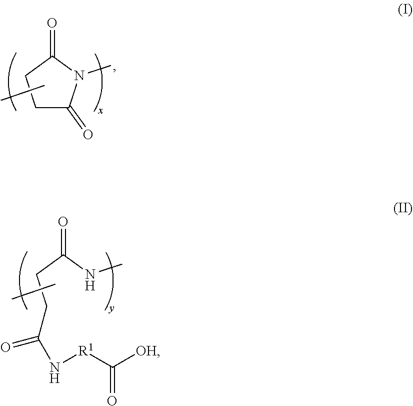
wherein in formulas (I) and (II), x is an integer ranging from 5 to 1000, y is an integer ranging from 5 to 1000, and R¹ is selected from the group consisting of a C₁-C₂₀ straight chain alkyl group and a C₂-C₂₀ branched chain alkyl group, and
wherein the second repeating unit is present in an amount ranging from 1 mol % to 90 mol % based on 100 mol % of the first repeating unit.

2. The polysuccinimide derivative as claimed in claim 1, wherein R¹ is selected from the group consisting of a C₅-C₂₀ straight chain alkyl group and a C₅-C₂₀ branched chain alkyl group.

3. The polysuccinimide derivative as claimed in claim 1, wherein the second repeating unit is present in an amount ranging from 5 mol % to 25 mol % based on 100 mol % of the first repeating unit.

4. A nanomaterial, comprising:
a plurality of nanoparticles, wherein under a pH of not greater than 6, each of the nanoparticles includes a hydrophobic substance and a carrier which is made from the polysuccinimide derivative as claimed in claim 1 and which encloses the hydrophobic substance.

5. The nanomaterial as claimed in claim 4, wherein the hydrophobic substance is selected from the group consisting of a hydrophobic drug and a hydrophobic dye.

6. The nanomaterial as claimed in claim 5, wherein the hydrophobic drug is an antibiotic.

7. The nanomaterial as claimed in claim 4, wherein the nanoparticles each has an average particle size ranging from 20 nm to 1000 nm.

8. The nanomaterial as claimed in claim 4, wherein the carrier has an average particle size ranging from 20 nm to 1000 nm.
